(19) United States
(12) Patent Application Publication
(10) Pub. No.: US 2022/0193155 A1
(43) Pub. Date: Jun. 23, 2022

(54) MICROBIAL COMPOSITIONS AND METHODS FOR GREATER TOLERABILITY AND PROLONGED SHELF LIFE

(71) Applicant: Pendulum Therapeutics, Inc., San Francisco, CA (US)

(72) Inventors: Andrew T. Cheng, San Francisco, CA (US); Marcus F. Schicklberger, San Francisco, CA (US); David Moreno, San Francisco, CA (US); Brendon Stoneburner, San Francisco, CA (US); Jaime Hernandez, San Francisco, CA (US)

(21) Appl. No.: 17/604,284

(22) PCT Filed: Apr. 21, 2020
(86) PCT No.: PCT/US2020/029110

§ 371 Date: Oct. 15, 2021

Related U.S. Application Data
(60) Provisional application No. 62/836,929, filed on Apr. 22, 2019.

(51) Int. Cl. A61K 35/745; A61K 47/12; A61K 47/26

(52) U.S. Cl. CPC A61K 35/745; A61K 47/26; A61K 47/12

(57) ABSTRACT

Provided herein are methods and compositions comprising microbial populations with increased tolerability and improved shelf life. Disclosed, herein, in some aspects, is a composition comprising at least one powdered microbial population, lactate, and trehalose. In some embodiments, the lactate is a lactate salt. In some embodiments, the lactate is sodium lactate. In some embodiments, the lactate and trehalose are present in sufficient amount to act as a cryoprotectant.

### CROSS REFERENCE

This application claims priority to U.S. Provisional Patent Application No. 62/836,929, filed Apr. 22, 2019, which is incorporated herein by reference in its entirety.

### BACKGROUND

In today's world, gut-oriented disorders can be common. Many of these disorders can involve inflammation or mal-digestive issues originating from, for example, dietary, hereditary, or allergic conditions. A variety of treatment approaches have been proposed for managing these issues, including, for example, changes to diet, reducing stress, taking medications, and supplements such as probiotics.

### BRIEF SUMMARY

Disclosed herein are microbial compositions and methods of producing such compositions that have improved properties, including but not limited to enhanced tolerability for subjects being administered such compositions, as well as improved shelf life and storability.

Disclosed, herein, in some aspects, is a composition comprising at least one powdered microbial population, lactate, and trehalose.

In some embodiments, the lactate is a lactate salt. In some embodiments, the lactate is sodium lactate. In some embodiments, the lactate and trehalose are present in sufficient amount to act as a cryoprotectant. In some embodiments, the microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, the lactate and trehalose are present in an amount from 1% to 50% weight by volume. In some embodiments, the lactate and trehalose are present in at least 5% weight by volume. In some embodiments, the lactate and trehalose are present at about 20% weight by volume. In some embodiments, the microbial population is lyophilized. In some embodiments, the microbial population is viable. In some embodiments, the microbial population has a viability of at least 1×10\hat{ }5 CFU/g of the composition. In some embodiments, the composition is dairy-free. In some embodiments, the composition comprises substantially no animal products. In some embodiments, the composition comprises an effective amount of a preservative. In some embodiments, composition further comprises a desiccant. In some embodiments, the desiccant is selected from the group consisting of, silica gel, clay, and calcium sulfate. In some embodiments, the composition has a moisture content from about 2.8% to about 5.6%. In some embodiments, the composition is a pill, capsule, or tablet. In some embodiments, the pill, capsule or tablet is enterically-coated, or the pill, capsule, or tablet disintegrates to release its contents in the small intestine. In some embodiments, the microbial population maintains at least 50% viability at room temperature for at least 5 days or at least 7 days. In some embodiments, the microbial population maintains at least 40% viability at room temperature for at least 19 days or at least 42 days.

Disclosed herein, in some aspects, is a method of producing a microbial product, the method comprising: combining a microbial population with lactate and trehalose so as to create a microbial product.

In some embodiments, the method further comprises lyophilizing, spray drying, and/or freeze-drying the microbial population. In some embodiments, the lactate is a lactate salt. In some embodiments, the lactate is sodium lactate. In some embodiments, the lactate and trehalose are present in sufficient amount to act as a cryoprotectant. In some embodiments, the microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, the lactate and trehalose are present in an amount from 1% to 50% weight by volume. In some embodiments, the lactate and trehalose are present in at least 5% weight by volume. In some embodiments, the lactate and trehalose are present at about 20% weight by volume. In some embodiments, the microbial product is dairy-free. In some embodiments, the microbial product comprises substantially no animal products.

In some cases, provided are dry, powdered microbial compositions that provide improved tolerability for human consumption, which in some cases may not include animal products and/or dairy-derived components.

In some cases, provided herein are microbial compositions and processes for making such compositions, wherein such compositions have improved shelf life or storability.

In some embodiments, the compositions comprise at least one strain of interest. In some embodiments, the strain of interest is selected from the group consisting of Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and Faecalibacterium prausnitzii. In some embodiments, the strain of interest comprises an rRNA sequence comprising at least about 90% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, in the composition, at least one of microbial strain is lyophilized. In further embodiments, the strain of interest in the composition is viable

In some embodiments, the microbial compositions provided herein comprises at least one microbial population, wherein the microbial population is grown in a dairy free media. In some embodiments, the growth media is also free of animal-derived components, animal products, animal by-products, or a combination thereof. In some embodiments, the growth medium comprises peptones, sugars, vegetable extracts, or any combination thereof. In some embodiments, the growth medium is a plant-based or a yeast-based growth medium. In some embodiments, the harvesting the cultured strain of interest is performed when the concentration of the strain of interest is at least 10\hat{ }7 CFU/gram. In additional embodiments, at least a portion of the microbial population is viable.

In some embodiments, the population of microbes provided herein are combined in the composition with a cryoprotectant that is dairy free and/or free of animal-derived components, animal products, animal by-products, or a combination thereof. In some embodiments, the cryoprotectant comprises lactate, trehalose, or a combination thereof. In some embodiments, the dry powder microbial composition comprises the cryoprotectant at 5% weight by volume. In some embodiments, the cryoprotectant can be selected from lactate or derivatives thereof, trehalose, Polyvinyl pyrrolidone (PVP), methylcellulose, tapioca or combinations thereof. In some embodiments, the dry powder microbial composition comprises the cryoprotectant at 1%-50% weight by volume. In some embodiments, the cryoprotectant is a combination of sodium lactate and trehalose. In some embodiments, the cryoprotectant is a combination of sodium lactate and trehalose at 5% weight by volume. In additional embodiments, at least a portion of the cryoprotected microbial population is viable.

In some embodiments, the dry powder microbial composition has a moisture content from about 2.8% to about 5.6%. In some embodiments, the composition includes a desiccant. In embodiments, the amount of the desiccant is such that the moisture content of the composition is less than 6%, preferably between 2.8% and 5.6%. In some embodiments, the desiccant is added after the microbes are lyophilized into fine powder form. Commonly used desiccants include clays, silica gels, and calcium sulfate. The desiccant can be added directly or indirectly into the powdered microbial composition. In some embodiments, the desiccant is placed in a sachet or a pouch or is separated by a membrane from the dry powdered microbial composition.

In some aspects, the present disclosure provides a method, comprising the steps of: (a) providing a strain of interest; (b) culturing the strain of interest in a growth medium; (c) harvesting the cultured strain of interest; and (d) formulating the cultured strain of interest as a dry powder microbial composition; wherein the culturing, the harvesting, and the formulating are carried out in a manner that introduces substantially no animal products into the dry powder microbial composition.

In some embodiments, a formulation comprising at least one microbial population is disclosed. The formulation can be a dry powder composition. Formulating the cultured strain of interest as a dry powder microbial composition comprises lyophilization, spray-drying, freeze-drying, or a combination thereof. In some embodiments, the formulating the cultured strain of interest as a dry powder microbial composition comprises cryoprotecting the cultured strain of interest using a cryoprotectant, wherein the cryoprotectant is animal product-free. In some embodiments, the formulating the cultured strain of interest as a dry powder microbial composition comprises cryoprotecting the cultured strain of interest using a cryoprotectant, wherein the cryoprotectant is dairy-free.

In some embodiments, the strain of interest comprises an rRNA sequence comprising at least about 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii.

In some aspects, the present disclosure provides a method for treating a gut-oriented disorder in a subject, comprising: administering to the subject a composition comprising a dried microbial formulation comprising at least one microbial population selected to mitigate the gut-oriented disorder in the subject, wherein the dried microbial formulation is substantially free of animal products.

In some embodiments, the gut-oriented disorder is Crohn's disease, diarrhea, irritable bowel syndrome, or inflammatory bowel disease, or gastritis. In some embodiments, the dried microbial formulation is in the form of a powder. In some embodiments, the dried microbial formulation comprises one or more viable strains selected from the group consisting of Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. In some embodiments, the administering comprises administering by an oral, parenteral, or suppository route. In some embodiments, the composition comprises less than 0.05% animal products. In some embodiments, the composition comprises less than 0.05% dairy-derived components. In some embodiments, the composition is administered to the subject on a daily basis. In some embodiments, the composition comprises at least 10\hat{ }6 CFU of the at least one microbial population per gram of the composition. In some embodiments, the composition is administered to the subject as a tablet, a capsule, a pill, a bar, or a suspension, wherein the suspension comprises the powder mixed with saline.

In some aspects, the present disclosure provides a method, comprising the steps of: (a) providing a strain of interest; (b) culturing the strain of interest in a growth medium; (c) harvesting the cultured strain of interest; and (d) formulating the cultured strain of interest as a dry powder microbial composition; wherein the culturing, the harvesting, and the formulating are carried out in a manner that introduces substantially no dairy-derived components into the dry powder microbial composition.

In some aspects, the present disclosure provides a method for treating a gut-oriented disorder in a subject, comprising administering to the subject a composition comprising a dried microbial formulation comprising at least one microbial population selected to mitigate the gut-oriented disorder in the subject, wherein the dried microbial formulation is substantially free of dairy-derived components.

In some embodiments, the gut-oriented disorder is Crohn's disease, diarrhea, irritable bowel syndrome, or inflammatory bowel disease, or gastritis. In some embodiments, the dried microbial formulation is in the form of a powder. In some embodiments, the dried microbial formulation comprises one or more viable strains selected from the group consisting of Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. In some embodiments, the administering comprises administering by an oral, parenteral, or suppository route. In some embodiments, the composition comprises less than 0.05% animal products. In some embodiments, the composition comprises less than 0.05% dairy-derived components. In some embodiments, In some embodiments, the composition is administered to the subject on a daily basis. In some embodiments, the composition comprises at least 10\hat{ }6 CFU of the at least one microbial population per gram of the composition. In some embodiments, the composition is administered to the subject as a tablet, a capsule, a pill, a bar, or a suspension, wherein the suspension comprises the powder mixed with saline.

In some aspects, the present disclosure provides a process of manufacturing a microbial composition, comprising: (a) providing a strain of interest; (b) culturing the strain of interest in a growth medium; (c) harvesting the strain of interest from the growth medium; and (d) formulating the strain of interest into a composition suitable for administration to a subject; wherein the culturing, the harvesting, and the formulating do not comprise use of an animal product.

In some embodiments, the composition is formulated for oral delivery, parenteral delivery, or suppository delivery. In some embodiments, the composition is formulated as a pill, a capsule, a tablet, a bar, or an effervescent powder.

In some aspects, the present disclosure provides a process of manufacturing a microbial composition, comprising: (a) providing a strain of interest; (b) culturing the strain of interest in a growth medium; (c) harvesting the strain of interest from the growth medium; and (d) formulating the strain of interest into a composition suitable for administration to a subject; wherein the culturing, the harvesting, and the formulating do not comprise use of a dairy-derived component.

In some embodiments, the composition is formulated for oral delivery, parenteral delivery, or suppository delivery. In some embodiments, the composition is formulated as a pill, a capsule, a tablet, a bar, or an effervescent powder.

In some aspects, the present disclosure provides a composition for treating a subject with a gut-oriented disorder, the composition comprising a viable microbial population formulated as a dry powder, wherein the composition is substantially free of animal products, wherein the composition provides greater tolerability in the subject as compared to compositions comprising animal products.

In some embodiments, the gut-oriented disorder is Crohn's disease, diarrhea, irritable bowel syndrome, or inflammatory bowel disease, or gastritis. In some embodiments, the viable microbial population comprises Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, the composition comprises less than 0.05% animal products. In some embodiments, the composition comprises less than 0.05% dairy-derived components. In some embodiments, the composition is in unit dosage form, wherein the unit dose comprises at least 10\hat{ }6 CFU of the viable microbial population. In some embodiments, the composition is formulated as a tablet, a capsule, a pill, a bar, or a suspension, wherein the suspension comprises the powder mixed with saline.

In some aspects, the present disclosure provides a composition for treating a subject with a gut-oriented disorder, the composition comprising a viable microbial population formulated as a dry powder, wherein the composition is substantially free of dairy-derived components, wherein the composition provides greater tolerability in the subject as compared to compositions comprising dairy-derived components.

In some embodiments, the gut-oriented disorder is Crohn's disease, diarrhea, irritable bowel syndrome, or inflammatory bowel disease, or gastritis. In some embodiments, the viable microbial population comprises Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, the composition comprises less than 0.05% animal products. In some embodiments, the composition comprises less than 0.05% dairy-derived components. In some embodiments, the composition is in unit dosage form, wherein the unit dose comprises at least 10\hat{ }6 CFU of the viable microbial population. In some embodiments, the composition is formulated as a tablet, a capsule, a pill, a bar, or a suspension, wherein the suspension comprises the powder mixed with saline.

In some aspects, the present disclosure provides a method of producing a microbial composition suitable for consumption by a subject, comprising: (a) providing an isolated microbe; (b) culturing the isolated microbe in a growth medium to provide a cultured microbe; (c) harvesting the cultured microbe from the growth medium; and (d) formulating the cultured microbe as a dry powder, thereby producing the microbial composition; wherein the microbial composition is stable at room temperature.

In some embodiments, the isolated microbe is selected from the group consisting of Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. In some embodiments, the growth medium comprises peptones, yeast, glucose, or a combination thereof. In some embodiments, the growth medium is a plant-based growth medium or a yeast-based growth medium. In some embodiments, the culturing, the harvesting, and the formulating are performed with substantially no animal-derived components. In some embodiments, the culturing, the harvesting, and the formulating are performed with substantially no animal and/or dairy-derived products. In some embodiments, the harvesting is performed when the concentration of the cultured microbe in the growth medium is at least 10\hat{ }7 CFU/gram. In some embodiments, the microbial composition has a moisture content from about 2.8% to about 5.6%. In some embodiments, the formulating comprises lyophilization, spray drying, freeze-drying, or a combination thereof. In some embodiments, the formulating comprises cryoprotecting using a cryoprotectant that is animal product-free. In some embodiments, the formulating comprises cryoprotecting using a cryoprotectant that is dairy-free. In some embodiments, the cryoprotectant comprises lactate, trehalose, or a combination thereof. In some embodiments, the microbial composition is stable at temperatures between about 4° C. and about 35° C. for at least 30 days.

In some aspects, the present disclosure provides a process of manufacturing a microbial composition, comprising the steps of: (a) providing an isolated microbe; (b) culturing the isolated microbe in a growth medium to provide a cultured microbe; (c) harvesting the cultured microbe from the growth medium; and (d) formulating the cultured microbe in a microbial composition suitable for administration to a subject; wherein the microbial composition is stable for at least 30 days.

In some embodiments, the isolated microbe is selected from the group consisting of Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. In some embodiments, the growth medium comprises peptones, yeast, glucose, or a combination thereof. In some embodiments, the growth medium is a plant-based growth medium or a yeast-based growth medium. In some embodiments, the culturing, the harvesting, and the formulating are performed with substantially no animal-derived components. In some embodiments, the culturing, the harvesting, and the formulating are performed with substantially no animal and/or dairy-derived products. In some embodiments, the harvesting is performed when the concentration of the cultured microbe in the growth medium is at least 10\hat{ }7 CFU/gram. In some embodiments, the microbial composition has a moisture content from about 2.8% to about 5.6%. In some embodiments, the formulating comprises lyophilization, spray drying, freeze-drying, or a combination thereof. In some embodiments, the formulating comprises cryoprotecting using a cryoprotectant that is animal product-free. In some embodiments, the formulating comprises cryoprotecting using a cryoprotectant that is dairy-free. In some embodiments, the cryoprotectant comprises lactate, trehalose, or a combination thereof. In some embodiments, the microbial composition is stable at temperatures between about 4° C. and about 35° C. for at least 30 days.

In some aspects, the present disclosure provides a composition comprising at least one viable microbial population, wherein the composition comprises an animal product-free cryoprotectant, wherein the at least one viable microbial population maintains at least 50% viability at room temperature for at least 5 days in the composition.

In some embodiments, the at least one viable microbial population comprises Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, or a combination thereof. In some embodiments, the at least one viable microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, the composition is substantially animal product-free. In some embodiments, the composition is substantially free of dairy-derived components. In some embodiments, the at least one viable microbial population maintains at least 50% viability at a temperature of about 25° C. for at least 30 days. In some embodiments, the composition is in a unit dosage form comprising at least 10\hat{ }6 CFU of the at least one viable microbial population, wherein at least 50% of the unit dose is stable at about 25° C. for at least 5 days. In some embodiments, the at least one viable microbial population maintains at least 50% viability at room temperature for at least 30 days in the composition. In some embodiments, the at least one viable microbial population maintains at least 80% viability at room temperature for at least 5 days in the composition. In some embodiments, the at least one viable microbial population maintains at least 90% viability at room temperature for at least 5 days in the composition. In some embodiments, the reduction in viability of the microbial population between day 1 and day 5 is less than 0.05%.

In some aspects, the present disclosure provides a composition comprising at least one viable microbial population, wherein the composition comprises a dairy-free cryoprotectant, wherein the at least one viable microbial population maintains at least 50% viability at room temperature for at least 5 days in the composition.

In some embodiments, the at least one viable microbial population comprises Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, or a combination thereof. In some embodiments, the at least one viable microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii. In some embodiments, the composition is substantially animal product-free. In some embodiments, the composition is substantially free of dairy-derived components. In some embodiments, the at least one viable microbial population maintains at least 50% viability at a temperature of about 25° C. for at least 30 days. In some embodiments, the composition is in a unit dosage form comprising at least 10\hat{ }6 CFU of the at least one viable microbial population, wherein at least 50% of the unit dose is stable at about 25° C. for at least 5 days. In some embodiments, the at least one viable microbial population maintains at least 50% viability at room temperature for at least 30 days in the composition. In some embodiments, the at least one viable microbial population maintains at least 80% viability at room temperature for at least 5 days in the composition. In some embodiments, the at least one viable microbial population maintains at least 90% viability at room temperature for at least 5 days in the composition. In some embodiments, the reduction in viability of the microbial population between day 1 and day 5 is less than 0.05%.

In some aspects, the present disclosure provides a method of treating a gut-oriented disorder in a subject in need thereof, the method comprising: (a) providing a composition comprising at least one viable microbial population, wherein the composition comprises an animal product-free cryoprotectant, wherein the at least one viable microbial population maintains at least 50% viability at room temperature for at least 5 days in the composition; and (b) administering the composition to the subject.

In some embodiments, the composition is administered as a pill, capsule, or tablet. In some embodiments, the pill, capsule or tablet is enterically-coated, wherein the pill, capsule, or tablet is administered orally, wherein the pill, capsule, or tablet disintegrates to release its contents in the small intestine of the subject. In some embodiments, the gut-oriented disorder is diarrhea, gastritis, inflammation of the gut, Crohn's disease, irritable bowel syndrome, inflammatory bowel disease, or dysbiosis. In some embodiments, the gut-oriented disorder is a comorbidity of dysbiosis selected from the group consisting of liver disease, kidney disease, obesity, diabetes, cardiovascular disease, allergic disease, rheumatoid arthritis, neurologic disorder, and cancer.

In some aspects, the present disclosure provides a method of treating a gut-oriented disorder in a subject in need thereof, the method comprising: (a) providing a composition comprising at least one viable microbial population, wherein the composition comprises a dairy-free cryoprotectant, wherein the at least one viable microbial population maintains at least 50% viability at room temperature over at least 5 days in the composition; and (b) administering the composition to the subject.

In some embodiments, the composition is administered as a pill, capsule, or tablet. In some embodiments, the pill, capsule or tablet is enterically-coated, wherein the pill, capsule, or tablet is administered orally, wherein the pill, capsule, or tablet disintegrates to release its contents in the small intestine of the subject. In some embodiments, the gut-oriented disorder is diarrhea, gastritis, inflammation of the gut, Crohn's disease, irritable bowel syndrome, inflammatory bowel disease, or dysbiosis. In some embodiments, the gut-oriented disorder is a comorbidity of dysbiosis selected from the group consisting of liver disease, kidney disease, obesity, diabetes, cardiovascular disease, allergic disease, rheumatoid arthritis, neurologic disorder, and cancer.

### BRIEF DESCRIPTION OF THE DRAWINGS

FIG. 1 illustrates comparative viability over time of an illustrative composition of the disclosure.

FIG. 2A shows the percent viability of a microbial composition when freeze-dried in the presence of lactate-trehalose cryoprotectant vs a cryoprotectant comprising skim milk over a period of 42 days.

FIG. 2B provides the normalized data used to generate FIG. 2A.

FIG. 3 illustrates a comparative study of relative viability of a microbial composition at room temperature over days when tested with various cryoprotectants.

### DETAILED DESCRIPTION

### I. General

Various treatment approaches have been proposed for managing gut-related disorders, including, for example, changes to diet, reducing stress, taking medications, and supplements such as probiotics. In some cases, it has been proposed to treat or modulate the intensity of such disorders by adjusting the make-up of the microbial populations within the gut. For example, Xifaxin is an antibiotic drug marketed for treatment of IBS that selectively targets certain undesirable bacteria in the gut, aiming to restore a healthy balance of bacteria. Conversely, other approaches have proposed supplementing the desirable microbial populations in the gut through administration of probiotics (desirable bacteria), synbiotics (food sources for desirable bacteria and bacterial populations) or pharmabiotics (pharmaceuticals that comprise prebiotics and probiotics). Each of these methods is suggested to increase the diversity, quantity, or activity of gut-located “good bacteria” by directly administering “good bacteria”, promoting the expansion of “good bacteria”, or decreasing the size of competing microbial populations. The optimized, diverse microbial populations the treatment methods seek to establish in the gut are thought to reduce inflammation and promote a healthy bowel. Although such treatment approaches have been proposed, significant challenges remain regarding their implementation. The present disclosure addresses these and many other needs.

As used in the specification and claims, the singular forms “a”, “an,” and “the” can include plural references unless the context clearly dictates otherwise. For example, the term “a sample” includes a plurality of samples, including mixtures thereof.

The terms “microbes” and “microorganisms” can be used interchangeably herein and can refer to bacteria, archaea, eukaryotes (e.g. protozoa, fungi, yeast), and viruses, including bacterial viruses (i.e. phage).

The term “microbiome”, “microbiota”, and “microbial habitat” can be used interchangeably herein and can refer to the ecological community of microorganisms that live on or in a subject's body. The microbiome can be comprised of commensal, symbiotic, and/or pathogenic microorganisms. Microbiomes can exist on or in many, if not most parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

The term “prebiotic” as used herein can be a general term to refer to chemicals and/or ingredients that can affect the growth and/or activity of microorganisms in a host (e.g. can allow for specific changes in the composition and/or activity of the microbiome). Prebiotics can confer a health benefit on the host. Prebiotics can be selectively fermented, e.g. in the colon. Some non-limiting examples of prebiotics can include: complex carbohydrates, complex sugars, resistant dextrins, resistant starch, amino acids, peptides, nutritional compounds, biotin, polydextrose, fructooligosaccharide (FOS), gal actooligosaccharides (GOS), inulin, lignin, psyllium, chitin, chitosan, gums (e.g. guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, and xylooligosaccharides (XOS). Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. capsule or dietary supplement).

The term “probiotic” as used herein can mean one or more microbes which, when administered appropriately, can confer a health benefit on the host or subject. Some non-limiting examples of probiotics include: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof.

The terms “individual” and “subject” are used interchangeably and can include mammals, humans, birds, reptiles, fishes, amphibians, arthropods, and all other animal species.

The term “animal product” can refer to a component derived from, isolated from, or purified from one or more parts of an animal's body. Non-limiting examples of animal products include components derived from, isolated from, or purified from an animal carcass, shell, bone, skin, tissue, meat, cartilage, horn, hoof, organ, fat, flesh, or blood. Animal products can include, for example, meat digests, meat infusions, heart extracts, brain extracts, sera, blood or other blood-derived components, animal-derived proteins, animal-derived immunoglobulins, isinglass, and rennet. In some cases, an animal product described herein can comprise a sugar, lipid, protein, carbohydrate, sterol, nucleic acid, vitamin, or mineral derived from, isolated from, or purified from one or more parts of an animal's body. The term “animal product-free” can refer to a composition that is substantially free of or completely free of animal products.

The term “animal by-product” can refer to a substance originating from an animal. Non-limiting examples of animal by-products include dairy, eggs, honey, and parts and derivatives thereof. In some cases, an animal by-product can include compositions prepared by processing one or more animal by-products, including milk, compounds derived from or isolated from animal milk, eggs, compositions derived from or isolated from eggs, honey, and other animal by-products. In some cases, an animal by-product described herein can comprise a sugar, lipid, protein, carbohydrate, sterol, nucleic acid, vitamin, or mineral obtained from an animal source. The term “animal by-product-free” can refer to a composition that is substantially free of or completely free of animal by-products.

A protein that is considered an animal product or animal by-product, as described herein, can comprise a protein or protein component from a multicellular non-plant and non-yeast eukaryote. In some cases, animal proteins and animal protein components can be distinguished from non-animal proteins, for example, small polypeptides and oligopeptides obtainable from plants (usually about 10-30 amino acids in length), such as the soy bean, or from lower eukaryotes, such as yeast.

The term “animal-derived” can refer to a component or ingredient that is derived from an animal source. It can include any component, including an intermediary or a derivative that originates from, is extracted from or is isolated from a non-human animal, whether living or dead. Animal-derived can include animal products and animal by-products. The term “free of animal-derived components” can refer to a composition that is substantially free of or completely free of animal-derived components.

In some cases, a composition described herein can be free or substantially-free of animal products, animal by-products, animal-derived components, or a combination thereof. In some cases, a composition described herein can be cultured and processed without any animal products, animal by-products, animal-derived components, or a combination thereof. In some cases, a process can be carried out such that no animal products or derivatives thereof are introduced into the composition. In such cases, reagents, such as solvents, lyophilization reagents, buffers, mixtures, desiccants, cryoprotectants, and other products contacting the composition can be free or substantially free of animal products. In some cases, a process can be carried out such that no animal by-products or derivatives thereof are introduced into the composition. In such cases, reagents, such as solvents, lyophilization reagents, buffers, mixtures, desiccants, cryoprotectants, and other products contacting the composition can be free or substantially-free of animal by-products. In some cases, a process can be carried out such that no animal-derived components or derivatives thereof are introduced into the composition. In such cases, reagents, such as solvents, lyophilization reagents, buffers, mixtures, desiccants, cryoprotectants, and other products contacting the composition can be free or substantially free of animal-derived components.

In some cases, substantially free of animal products can be having less than about %, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001% of animal products (for example, weight by volume, volume by volume, or weight by weight).

In some cases, substantially-free of animal by-products can be having less than about 1%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001% of animal by-products (for example, weight by volume, volume by volume, or weight by weight).

In some cases, substantially free of animal-derived components can be having less than about 1%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001% of animal-derived components (for example, weight by volume, volume by volume, or weight by weight).

The term “dairy-derived” can refer to any product that originates from milk, including, for example, milk proteins, milk solids, whey proteins, or milk sugars (e.g., lactose). The term “dairy-free” can refer to any product or composition that is free or substantially-free of dairy-derived components.

In some cases, substantially free of dairy-derived components can be having less than about 1%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001% of dairy-derived components (for example, weight by volume, volume by volume, or weight by weight). In some cases, a composition described herein can be free or substantially-free of dairy-derived components. In some cases, a composition described herein can be grown and processed without any dairy-derived components. In some cases, a process can be carried out such that no dairy-derived components or derivatives thereof are introduced into the composition. In such cases, reagents, such as solvents, lyophilization reagents, buffers, mixtures, desiccants, cryoprotectants, and other products contacting the composition can be free or substantially free of dairy-derived components.

In some cases, a composition of the present disclosure may be a vegan composition in the sense that it can be, for example, animal product-free and dairy-free, or animal product-free, animal by-product-free, and dairy-free.

In some cases, a composition of the disclosure can be substantially free of animal products but may not be dairy-free.

In some cases, a composition of the disclosure can be dairy-free but may comprise an animal product.

The term “gut-oriented disorder” can refer to any disorder associated directly or indirectly with an underlying disorder affecting the gut or intestine. Non-limiting examples of gut-oriented disorders include diarrhea, inflammation of the gut, Crohn's disease, irritable bowel syndrome (IBS), inflammatory bowel disease (IBD), dysbiosis, gastritis, dysbiosis-associated disorders, and comorbidities thereof.

### II. Overview

There can be trillions of microbes residing in and on a body of an individual. The intestine (or gut) alone constitutes a significant quantity and diversity of microbes. These microbes can belong to a variety of categories including, for example, Bacteroides, Clostridium, Faecalibacterium, Eubacterium, Ruminococcus, Peptococcus, Peptostreptococcus, and Bifidobacterium. Different microbes can contribute to the process of digestion, and/or can contribute to the overall health of an individual, for example, by improving immunity, enriching nutrient absorption, maintaining the pH and other gut-environmental conditions at optimal levels, or even enriching mental health.

Imbalances in the populations of these microbes in the gut can result in a malfunction of any one or more of these avenues of health, and can affect the overall well-being of an individual. This imbalance, sometimes referred to as “dysbiosis,” can be caused by dietary changes, hormonal changes, medicinal side-effects, stress or anxiety, or even hereditary and/or genetic factors.

While dysbiosis may be characterized by relatively superficial health indicators, e.g., a change of bowel movement in quality and frequency, gas, bloating, or abdominal discomfort, gut dysbiosis may also be associated with more severe, pathological conditions. Dysbiosis-associated disorders or comorbidities of dysbiosis can include, for example, liver diseases (such as non-alcoholic steatohepatitis (“NASH”), or non-alcoholic fatty liver disease (“NAFLD”)), kidney diseases, obesity, diabetes, cardiovascular diseases, allergic diseases, rheumatoid arthritis, neurologic disorders, and cancer.

Non-limiting examples of gut-associated disorders or comorbidities thereof can include inflammatory and other conditions, Type 2 Diabetes Mellitus (T2DM), prediabetes, preterm labor, chronic fatigue syndrome, skin conditions such as acne, allergies, autism, asthma, depression, hypertension, irritable bowel syndrome, metabolic syndrome, obesity, lactose intolerance, oral thrush, ulcerative colitis, drug metabolism, vaginosis, atopic dermatitis, psoriasis, Type I Diabetes Mellitus (T1DM), Multiple Sclerosis, neurological disorders such as Parkinson's disease, Clostridium difficile infection, Inflammatory Bowel Disease, Crohn's Disease, heart disease, diabetic foot ulcers, bacteremia, infantile colic, cancer, cystic fibrosis, multiple sclerosis, urinary tract infection, radiation enteropathy, drug metabolism, dental cavities, and halitosis.

In some cases, the gut-associated disorder is diabetes, for example, Type II diabetes. In some cases, the gut-associated disorder is irritable bowel syndrome. In some cases, the gut-associated disorder is inflammatory bowel disease. In some cases, the gut-associated disorder is Crohn's disease.

In some cases, the subject has a gut-associated disorder (e.g., IBS, IBD, and/or diabetes) and also has a food allergy.

In some cases, the subject has a gut-associated disorder (e.g., IBS, IBD, and/or diabetes) and is lactose intolerant.

Inflammatory gut disorders can include irritable bowel syndrome (MS) and inflammatory bowel disease (IBD). Irritable Bowel Syndrome (MS) can be a painful and sometimes debilitating disorder that, based on its symptoms, classifies into one of four subforms IBS-C (constipation predominant), IBS-D (diarrhea predominant), IBS-M (mixed IBS—both—constipation and diarrhea), and IBS-U (unclear IBS). The symptoms common to all IBS forms can include pain related to defecation, change in stool frequency, and a change in the appearance of the stool, based on Rome IV criteria.

Inflammatory bowel disease (IBD) can involve chronic inflammation of all or part of the digestive tract. IBD can lead to ulcerative colitis and/or Crohn's disease. IBD can be painful and debilitating, and can sometimes lead to life-threatening complications. There can be various immunological, microbial, environmental, nutritional, and genetic factors that may contribute to the pathogenesis and severity of the disorder.

Gut-oriented disorders, such as IBS or IBD, can be marked and/or exacerbated by increased sensitivity to dietary components. For example, those suffering from IBS and/or IBD can also suffer from intolerance of food components, such as lactose intolerance, which may be associated with the underlying condition, or may exacerbate the condition, e.g., by causing additional abdominal distress.

Gut-oriented disorders such as those disclosed herein may be treated, ameliorated, or mitigated in those afflicted through the administration of microbial compositions that can reinstate, re-populate, or supplement good microbial strains in the gut. Administration of such composition can remove and/or prevent growth of non-favorable or problem causing microbes in the gut, or combinations of these. For instance, IBS patients can present with a reduced number of Lactobacillus and Bifidobacteria (also called lactic acid bacteria) species in their gut, compared to normal individuals. The lack of these strains in the gut of IBS patients can lead to proliferation of other strains of microbes that, in increased numbers, may have adverse effects on the gut.

Because of their high concentrations of lactic acid bacteria, it has become common practice to rely on consumption of yogurt, cheeses, and other fermented dairy products as a restorative treatment for gut-oriented disorders (e.g., IBS, IBD, gut dysbiosis). However, consumption of dairy based products can be problematic for these afflicted populations; as they can often have lactose intolerance or other food tolerability issues.

In cases of inflammatory gut disorders (e.g., IBS, IBD), dairy based ingredients or products can act as an irritant. For people who suffer from these disorders or are lactose-intolerant (associated often with IBD, IBS), the use of dairy-containing products may result in exacerbation of the condition.

The compositions, processes, and methods described herein are directed to providing microbial compositions for the treatment, amelioration, or mitigation of gut dysbiosis and other conditions, without compounding the conditions through the inclusion of potentially problem causing components. Moreover, these methods, compositions and processes can also provide additional benefits, including, for example, improved maintenance of microbial viability, simplified dosing, longer shelf life, and enhanced tolerability in subjects.

Another challenge can exist in being able to deliver sufficient quantities of viable microbes to the gut of a subject, and in some cases, being able to produce and supply viable microbial compositions to those subjects through a conventional commercial pathway. Accordingly, it may be important for such a composition to be able to maintain sufficient viability of the microbes until the composition is administered to the individual. For example, in some cases, it may be desirable to administer an effective amount of a microbial composition in as few doses as possible. In some cases, it may be desirable to administer an effective amount of a microbial composition without introducing excess adjunct materials associated with those compositions, e.g., materials that may present tolerability issues. Accordingly, ensuring that the compositions maintain optimal viability from the time they are prepared until they are administered (shelf life) may be important. Generally, viability of these compositions can be maintained by storing and offering such products for sale in refrigerated or frozen conditions. This can incur extra packaging, transportation, and storage costs, and can also limit date of consumption of these products, as over time and with exposure to environmental conditions, the viability of the microbes may otherwise tend to decrease. Reduced viability of the microbes in an administered composition may adversely impact the efficacy of the composition when consumed, and higher doses may need to be administered to already delicate or compromised systems. Therefore, providing a stable, viable composition that has greater efficacy when consumed or administered to a suffering individual may be advantageous.

The present disclosure provides methods and compositions for treating, ameliorating, or mitigating gut-oriented disorders by administering a composition comprising stable, viable microbes. In some cases, the methods and the compositions can be free or substantially free of animal products, animal by-products, dairy-derived components, animal-derived components, or a combination thereof. The methods and compositions can have greater stability over time and/or when stored in non-refrigerated temperatures, e.g., greater than 4° C.

### III. Improved Compositions and Methods

A. Removal of Problematic Components

Described herein are compositions, methods of making such compositions, and uses of such compositions in treating, ameliorating, or mitigating health disorders in a subject. For example, microbial compositions are provided that can substantially lack components that may cause additional problems or exacerbate conditions in a subject. In particular, an aspect of the disclosure relates to such compositions that can be produced without—or processed to remove—a host of components that can cause, be expected, or be reasonably believed to cause problems in different subjects.

By way of example, production of microbial compositions for use in treating, ameliorating, or mitigating, e.g., gut dysbiosis, can often utilize animal products, animal by-products, dairy-derived components, animal-derived components, or a combination thereof in the feeding, culturing or downstream processing of the microbes. For example, animal-derived components can be used in harvesting, cryopreserving, or formulating a microbial composition. Such animal-derived components may include, for example, animal products (e.g., animal based protein extracts or serum), or dairy-derived components (e.g., milk, milk solids, milk proteins, whey proteins, milk sugars, or the like).

Administering a microbial composition comprising animal products, animal by-products, dairy-derived components, or other animal-derived components to a subject can be problematic, for example, if the subject has delicate or compromised gut system, e.g., a subject suffering from lactose intolerance, a gut-oriented disorder, IBS, or IBD.

In some cases, the present disclosure provides microbial compositions that are free or substantially-free of animal products, animal by-products, dairy-derived components, or a combination thereof, e.g., microbial compositions that are free or substantially-free of animal products and free or substantially-free of dairy-derived components. In some cases, the present disclosure provides microbial compositions that are free or substantially-free of animal-derived components.

In some cases, the present disclosure provides methods for generating microbial compositions that are free or substantially-free of animal products, animal by-products, dairy-derived components, or a combination thereof, e.g., free or substantially-free of animal products and free or substantially-free of dairy-derived components. In some cases, the present disclosure provides methods for generating microbial compositions that are free or substantially-free of animal-derived components.

In some cases, provided herein are methods for generating microbial compositions that exhibit prolonged shelf life and improved viability, wherein the methods do not involve the use of animal products, animal by-products, dairy-derived components, animal-derived components, or a combination thereof. For example, the compositions and methods of producing those compositions, as disclosed herein, can be substantially or completely animal product-free or dairy-free. In some cases, one, multiple, or all of the steps in the process can be performed free or substantially free of animal products, animal by-products, dairy-derived components, animal-derived components, or a combination thereof.

Methods of the disclosure can comprise, for example, culturing, harvesting, cryoprotecting, lyophilizing, formulating, administering, or a combination thereof. In some cases, a selected microbe can be cultured in a growth medium. Animal products, and animal by-products, and dairy-derived components can be commonly used in microbial growth media. Blood serum, other blood-derived components, meat infusions, heart or brain extracts can be routinely employed in growing microbial cultures, for example, in growing Bacteroides, Prevotella, Fusobacterium, Clostridium, and Staphylococcus. Blood serum can be used as a source of hemin and other nutrients for high density growth of, for example, bacterial cultures, anaerobic bacterial cultures, or mammalian cell cultures.

In some embodiments, culturing a microbe of the disclosure can comprise culturing with a medium that is animal product-free, animal by-product-free, dairy-free, free of animal-derived components, or a combination thereof. Non-limiting examples of media that can be used include plant-derived (e.g., plant-based) growth media, yeast-derived (e.g., yeast-based) growth media, and synthetic media (e.g., chemically-defined media). Plant-derived or yeast-derived growth media can include, for example, plant-based peptone media or yeast-based peptone media, which can include vegetable-derived peptone(s), vitamins, glucose, salts of calcium, magnesium, potassium, and a fiber source. An exemplary culture medium of a composition as encompassed by the disclosure comprises peptones, sugars (e.g, dextrose), vegetable extracts, trace minerals and salts (e.g., salts of sodium, potassium, magnesium, calcium), and Tween 80.

A plant-based or yeast-based growth medium can be supplemented with components that are animal product-free, animal by-product-free, dairy-free, free of animal-derived components, or a combination thereof, for example, one or more vitamins, minerals (e.g., trace minerals), growth factors, carbohydrates (e.g., sugars), buffers, or salts. In some embodiments, a plant-based growth medium can be supplemented with components that are not plant-derived, but are animal product-free, animal by-product-free, dairy-free, free of animal-derived components, or a combination thereof, for example, one or more vitamins, minerals (e.g., trace minerals), growth factors, carbohydrates (e.g., sugars), buffers, or salts. In some embodiments, a yeast-based growth medium can be supplemented with components that are not yeast-derived, but are animal product-free, animal by-product-free, dairy-free, free of animal-derived components, or a combination thereof, for example, one or more vitamins, minerals (e.g., trace minerals), growth factors, carbohydrates (e.g., sugars), buffers, or salts.

Yeast-based or yeast-derived growth media can comprise an extract of a yeast, for example, a Saccharomyces species such as Saccharomyces cerevisiae. A yeast extract can comprise, for example, nitrogenous compounds, carbon, sulfur, trace nutrients, vitamin B complex, important growth factors, or a combination thereof. A yeast extract can be a hydrolyzed yeast extract (also called yeast peptone), or an autolyzed yeast extract.

In some cases, the media can be free of animal derived peptones, which can often be employed as a source of nitrogen in growth media. Commonly used peptones are made by cooking milk or meat products in acid, or by enzymatically treating milk or meat with trypsin, pepsin, or other proteolytic enzymes to digest the protein to a mixture of amino acids, peptides, and polypeptides. However, in accordance with the processes and compositions described herein, peptones used herein can be animal product free, animal by-product-free, dairy-free, free of animal-derived components, or a combination thereof. Examples of such non-animal or vegetable peptones include, for example, plant and/or yeast derived peptones, such as HiVegPeptone(s). A medium that is animal product-free, animal by-product-free, dairy-free, free of animal-derived components, or a combination thereof can be a plant-based medium. A medium that is animal product-free, animal by-product-free, dairy-free, free of animal-derived components, or a combination thereof can be a yeast-based medium. A medium that is animal product-free, animal by-product-free, and dairy-free can be a vegan medium. A medium that is animal product-free, animal by-product-free, free of animal-derived components, and dairy-free can be a vegan medium.

The culturing process may be carried out in varying scales, e.g., from about 1 liter scale to upwards of tens of thousands of liters, depending upon the quantity of microbial composition and viability of the microbes in the composition, as desired. Culturing can begin with inoculation of the growth media with an initial inoculant of the microbial species. The initial inoculant can be of a single microbial species or a combination of microbial species. The optical density of the medium can be measured at time intervals ranging from, for example, 15 minutes to 24 hours, to determine the population or concentration of microbes in the medium. Once at an optimal concentration, as desired based on the strain(s) being grown in the medium and the composition formulation, the microbial species or combination of species can be recovered, stored, or harvested. A microbe can be recovered, stored, or harvested at any phase of growth, for example, early exponential phase, mid exponential phase, late exponential phase, or stationary phase.

During or after growth, the cultured microbe can be stored, harvested, stored then harvested, or harvested then stored. If the microbe is stored, it can be stored, for example, at room temperature, at a refrigerated temperature, or at a frozen temperature. The microbe can be harvested using any acceptable method. For example, the microbe can be pelleted. In some cases, a microbe can be resuspended in a storage medium, a formulating medium, a medium comprising a cryoprotectant, or another medium.

In some cases, it may be desirable to provide a high quantity of viable microbes in a dosage to an individual receiving the composition. High dosages can be facilitated by the ability to culture the microbes to a higher density. For example, by starting with higher concentrations of viable microbes in the culture, one can reduce processes and associated costs with converting the resulting microbes into a product form, e.g., purification, powdering, etc.

The optimal concentration of a microbial species can range from 10¹ to 10¹⁸ CFU (Colony forming units) per gram of the composition. Each species harvested can have a unique range of CFU(s) considered optimal for the composition. The concentration of a microbial species can be, for example, at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸ CFU per gram of a composition. In notable examples, the concentration can be at least 10⁸, at least 10⁹, at least 10¹⁰, or at least 10¹¹ CFU per gram of a composition.

Animal-derived components (e.g., animal products, animal by-products, or dairy-derived components) may also be incorporated into microbial compositions in other process steps. For example, formulating a microbial composition can comprise introducing additional components to stabilize the microbes, e.g., to improve and/or prolong viability of the microbes in the finished product. After a cultured microbial species is harvested, it may be optionally be converted from liquid microbial compositions into dry, powdered forms to preserve the viability of the microbes over longer periods and/or to facilitate formulation into an administrable format. Such conversion may be carried out by a number of processes, including spray drying, freeze drying (or lyophilization), fluid bed spray coating, or any of a variety of other drying techniques. In some cases, freeze drying processes can be preferred for preserving maximum viability for these microbial compositions both during the drying processes, and following drying, as a result of the significant reduction in water activity of the finished product.

During the freeze drying processes, components called “cryoprotectants” may be added to the composition to improve the viability of the microbial compositions during and after the freeze-drying process. Cryoprotectants can be employed in the lyophilization process to preserve and maintain protein and membrane structure, improve the survival of microbial cells during the freeze-drying process, and maintain the stability of the composition. Skim milk, or other dairy-derived components can be routinely used as cryoprotectants in these processes. However, in some cases, the present disclosure preempts the use of cryoprotectants that comprise dairy-derived components, animal products, animal by-products, animal-derived components, or a combination thereof. Instead, the cryoprotection can be achieved herein by use of cryoprotectants that are free of animal products, animal by-products, dairy-derived components, animal-derived components, or a combination thereof. For example, a cryoprotectant of the disclosure can comprise PVP, cellulose, methylcellulose, sucrose, polyethylene glycol, trehalose, lactate, tapioca, inulin, glycogen, glycerol, glucose, lactose, maltose, Me₂SO, betaine, sodium ascorbate, glutamate, maltodextrine, xylitol, or a combination thereof. In some cases, the cryoprotectant for use may comprise L-lactate, a lactate salt, trehalose, or a combination thereof.

The concentration of a cryoprotectant such as lactate and/or trehalose, can be, for example, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, or at least 50% weight by volume (or for example, volume by volume, or weight by weight) of the composition, as deemed suitable for the strain being enriched. A cryoprotectant can be added in the step before freeze-drying or any step before or during freeze-drying. The concentration of the cryoprotectant can be the concentration before freeze-drying, or at any step before, during, or after freeze drying.

In some embodiments, a concentration of lactate can be at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, or at least 50% of the composition, as deemed suitable for the strain being enriched (for example, weight by volume, volume by volume, or weight by weight).

In some embodiments, a concentration of lactate can be at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 30%, at most 40%, or at most 50% of the composition, as deemed suitable for the strain being enriched (for example, weight by volume, volume by volume, or weight by weight).

In some embodiments, a concentration of lactate can be about 1% to about 30%, about 1% to about 25%, about 1% to about 20%, about 1% to about 15%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 30%, about 2% to about 20%, about 2% to about 15%, about 2% to about 10%, about 2% to about 9%, about 2% to about 8%, about 2% to about 7%, about 2% to about 6%, about 2% to about 5%, about 2% to about 4%, about 2% to about 3%, about 3% to about 30%, about 3% to about 25%, about 3% to about 20%, about 3% to about 15%, about 3% to about 10%, about 3% to about 9%, about 3% to about 8%, about 3% to about 7%, about 3% to about 6%, about 3% to about 5%, about 3% to about 4%, about 5% to about 30%, about 5% to about 25%, about 5% to about 20%, about 5% to about 15%, about 5% to about 10%, about 5% to about 9%, about 5% to about 8%, about 5% to about 7%, about 5% to about 6%, about 7% to about 30%, about 7% to about 25%, about 7% to about 20%, about 7% to about 15%, about 7% to about 10%, about 10% to about 30%, about 10% to about 25%, about 10% to about 20%, or about 10% to about 15% of the composition, as deemed suitable for the strain being enriched (for example, weight by volume, volume by volume, or weight by weight).

In some embodiments, a concentration of trehalose can be at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, or at least 50% of the composition, as deemed suitable for the strain being enriched (for example, weight by volume, volume by volume, or weight by weight).

In some embodiments, a concentration of trehalose can be at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 30%, at most 40%, or at most 50% of the composition, as deemed suitable for the strain being enriched (for example, weight by volume, volume by volume, or weight by weight).

In some embodiments, a concentration of trehalose can be about 1% to about 30%, about 1% to about 25%, about 1% to about 20%, about 1% to about 15%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 30%, about 2% to about 20%, about 2% to about 15%, about 2% to about 10%, about 2% to about 9%, about 2% to about 8%, about 2% to about 7%, about 2% to about 6%, about 2% to about 5%, about 2% to about 4%, about 2% to about 3%, about 3% to about 30%, about 3% to about 25%, about 3% to about 20%, about 3% to about 15%, about 3% to about 10%, about 3% to about 9%, about 3% to about 8%, about 3% to about 7%, about 3% to about 6%, about 3% to about 5%, about 3% to about 4%, about 5% to about 30%, about 5% to about 25%, about 5% to about 20%, about 5% to about 15%, about 5% to about 10%, about 5% to about 9%, about 5% to about 8%, about 5% to about 7%, about 5% to about 6%, about 7% to about 30%, about 7% to about 25%, about 7% to about 20%, about 7% to about 15%, about 7% to about 10%, about 10% to about 30%, about 10% to about 25%, about 10% to about 20%, or about 10% to about 15% of the composition, as deemed suitable for the strain being enriched (for example, weight by volume, volume by volume, or weight by weight).

Examples of lactate salts include, but are not limited to, calcium lactate, sodium lactate, potassium lactate, and ammonium lactate.

In some embodiments, a desiccant is used to create a drier environment. The desiccant may be added after the microbes are lyophilized into fine powder form. Commonly used desiccants include clays, silica gels, and calcium sulfate. The desiccant may be added directly or indirectly into the powdered microbial composition. In some embodiments, the desiccant is placed in a sachet or a pouch or is separated by a membrane from the dry powdered microbial composition.

A microbe of the disclosure can be formulated into an administrable format, e.g., after culturing, harvesting, cryoprotecting, or a combination thereof. In some cases, the administrable format can be appropriate for oral administration, and in some cases the administrable format can be a format which is ready for administration with no further modification. In some cases, further modification can be performed prior to administration. For example, further modification can comprise grinding, dissolving, moistening, mixing, heating, cooling, freezing, thawing, adding to food, other modification, or any combination thereof.

In some cases, each step and all intermediary steps of culturing, harvesting, cryoprotecting, lyophilizing, and formulating can be carried out free or substantially-free of animal products, animal by-products, dairy-derived components, animal-derived components, or a combination thereof. In some cases, a subset of culturing, harvesting, cryoprotecting, lyophilizing, and formulating can be carried out free or substantially-free of animal products, animal by-products, dairy-derived components, animal-derived components, or a combination thereof.

Processes described herein may be applied to one or more microbes or microbial populations that are produced for use alone, or in combination with other microbial populations, in the compositions described herein.

Provided herein are microbial compositions, and methods of producing microbial compositions for use in treating, ameliorating, or mitigating health disorders, where one or more of the processing steps are carried out in order to produce a composition that can be free or substantially-free of a component, (e.g., an animal products, animal by-products, dairy-derived components, animal-derived components, or a combination thereof).

Substantially free or essentially free of a component (e.g., an animal product, animal by-product, dairy-derived component, or animal-derived component)) can mean that a composition can have, for example, less than about 5%, less than about 4%, less than about 3%, less than about 2%, less than about 1%, less than about 0.5%, less than about 0.1%, less than about 0.05%, less than about 0.01%, less than about 0.009%, less than about 0.008%, less than about 0.007%, less than about 0.006%, less than about 0.005%, less than about 0.004%, less than about 0.003%, less than about 0.002%, less than about 0.001%, less than about 0.0009%, less than about 0.0008%, less than about 0.0007%, less than about 0.0006%, less than about 0.0005%, less than about 0.0004%, less than about 0.0003%, less than about 0.0002%, less than about 0.0001%, or even less of that component by weight or volume of the composition (for example, weight by volume, or volume by volume, or weight by weight). In some instances, the microbial compositions may be devoid of the component. In some instances, the component may be undetectable or untraceable.

Provided herein, in some embodiments, are microbial compositions, and methods of producing microbial compositions for use in treating, ameliorating, or mitigating health disorders, where one or more of the processing steps are carried out in order to produce a composition that can be free or substantially free of animal products.

Substantially-free or essentially-free of animal products can mean that the composition can have, for example, less than 5% by weight or volume of the animal products, or preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal products by weight or volume of the composition (for example, weight by volume, volume by volume, or weight by weight). In some instances, the microbial compositions may be devoid of animal products. In some instances, the animal products may be undetectable or untraceable.

Also provided herein are microbial compositions, and methods of producing microbial compositions for use in treating, ameliorating, or mitigating health disorders, where one or more of the processing steps are carried out in order to produce a composition that can be free or substantially free of animal by-products.

Substantially-free or essentially-free of animal by-products can mean that the composition can have, for example, less than 5% by weight or volume of the animal by-products, or preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal by-products by weight or volume of the composition (for example, weight by volume, volume by volume, or weight by weight). In some instances, the microbial compositions may be devoid of animal by-products. In some instances, the animal by-products may be undetectable or untraceable.

Also provided herein are microbial compositions, and methods of producing microbial compositions for use in treating, ameliorating, or mitigating health disorders, where one or more of the processing steps are carried out in order to produce a composition that can be free or substantially free of dairy-derived components.

Substantially free or essentially free of dairy-derived components can mean that the composition can have, for example, less than 5% by weight or volume of the dairy-derived components, or preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less dairy-derived components by weight or volume of the composition (for example, weight by volume, volume by volume, or weight by weight). In some instances, the microbial compositions may be devoid of dairy-derived components. In some instances, the dairy-derived components may be undetectable or untraceable. Further provided herein are microbial compositions, and methods of producing microbial compositions for use in treating, ameliorating, or mitigating health disorders, where one or more of the processing steps are carried out in order to produce a composition that can be free or substantially free of animal products and dairy-derived components.

Substantially free of animal products and dairy-derived components can mean that the composition can have, for example, less than 5% by weight or volume of the animal products and dairy-derived components, preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal products and dairy-derived components by weight or volume of the composition (for example, weight by volume, volume by volume, or weight by weight). In some instances, the microbial compositions may be devoid of animal products and dairy-derived components. In some instances, the animal products and dairy-derived components can be undetectable or untraceable.

Further provided herein are microbial compositions, and methods of producing microbial compositions for use in treating, ameliorating, or mitigating health disorders, where one or more of the processing steps are carried out in order to produce a composition that can be free or substantially free of animal products and animal by-products.

Substantially free of animal products and animal by-products can mean that the composition can have, for example, less than 5% by weight or volume of the animal products and animal by-products, preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal products and animal by-products by weight or volume of the composition (for example, weight by volume, volume by volume, or weight by weight). In some instances, the microbial compositions may be devoid of animal products and animal by-products. In some instances, the animal products and animal by-products can be undetectable or untraceable.

Further provided herein are microbial compositions, and methods of producing microbial compositions for use in treating, ameliorating, or mitigating health disorders, where one or more of the processing steps are carried out in order to produce the composition that can be free or substantially free of animal by-products and dairy-derived components.

Substantially free of animal by-products and dairy-derived components can mean that the composition can have, for example, less than 5% by weight or volume of the animal by-products and dairy-derived components, preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal by-products and dairy-derived components by weight or volume of the composition (for example, weight by volume, volume by volume, or weight by weight). In some instances, the microbial compositions may be devoid of animal by-products and dairy-derived components. In some instances, the animal by-products and dairy-derived components can be undetectable or untraceable.

Further provided herein are microbial compositions, and methods of producing microbial compositions for use in treating, ameliorating, or mitigating health disorders, where one or more of the processing steps are carried out in order to produce a composition that can be free or substantially free of animal products, animal by-products and dairy-derived components.

Substantially free of animal products, animal by-products and dairy-derived components can mean that the composition can have, for example, less than 5% by weight or volume of the animal products, animal by-products and dairy-derived components, preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal products, animal by-products and dairy-derived components by weight or volume of the composition(for example, weight by volume, volume by volume, or weight by weight). In some instances, the microbial compositions may be devoid of animal products, animal by-products and dairy-derived components. In some instances, the animal products, animal by-products and dairy-derived components can be undetectable or untraceable.

Also provided herein are microbial compositions, and methods of producing microbial compositions for use in treating, ameliorating, or mitigating health disorders, where one or more of the processing steps are carried out in order to produce the composition that can be free or substantially free of animal-derived components.

Substantially free of animal-derived components can mean that the composition can have, for example, less than 5% by weight or volume of the animal-derived components, preferably less than 1%, less than 0.5%, less than 0.1%, less than 0.05%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001%, or even less animal-derived components by weight or volume of the composition (for example, weight by volume, volume by volume, or weight by weight). In some instances, the microbial compositions may be devoid of animal-derived components. In some instances, the animal-derived components can be undetectable or untraceable.

B. Improved Stability and Shelf Life of Compositions

In some cases, provided herein are microbial compositions, and preferably dry powder microbial compositions, that have enhanced properties, for example, related to their stability, storability, or shelf life. In some cases, the compositions may include those compositions that are free or substantially free of animal products, animal by-products, dairy-derived components, animal-derived components, or a combination thereof.

Improved stability and/or longer shelf life can comprise, for example, maintained viability of a microbe, strain of interest, or microbial composition stored under certain conditions over time, e.g. retention of viable CFU above a given percentage or concentration per unit measure over time. Improved stability and/or longer shelf life can comprise, for example, reduced loss of viability of a microbe, strain of interest, or microbial composition stored under certain conditions over time, e.g., loss of less than a given percentage of viable CFU over time, or loss of less than a given CFU per unit measure over time. Viability can be quantified, for example, by plating serial dilutions of a composition on agar plates to enumerate CFU, by quantification of ATP (e.g., via a luciferin-luciferase assay), or by use of reagents that distinguish live versus dead cells, for example, via membrane integrity (e.g., SYTO 9 and propidium iodide), membrane potential (e.g., DiOC₂(3)), or respiratory activity (e.g., 5-cyano-2,3-ditolyl tetrazolium chloride). Viability can refer to, for example, a number of viable cells, a concentration of viable cells, a percentage of total cells that are viable, or a calculated relative percentage of viable cells, for example, relative to before a processing step or before storage of a composition. In some cases, a microbial composition may exhibit a reduction in viability over time. For example, a microbial composition may exhibit a reduction in viability between the time the microbial composition is processed to an end use form, e.g., dried, powdered, encapsulated, or otherwise formulated and/or packaged, and the time the microbial composition is provided to a consumer, e.g., shipped, placed on store shelves or the like, or purchased/consumed. This reduction in viability may result from a number of factors, including, for example environmental factors to which the compositions are exposed, or a lack of growth factors in the composition to sustain microbial life after an expiry point. By way of example, microbial compositions in dried form may experience reductions in viability if they are exposed to moisture, which can result in viability decreases over time. Likewise, the presence of certain materials within a dried microbial composition may contribute to reduced viability over time.

In some embodiments, microbial compositions are provided comprising a specified moisture content, for example, a moisture content of less than 15%, less than 10%, less than 9%, less than 8%, less than 7.5%, less than 7.4%, less than 7.3%, less than 7.2%, less than 7.1%, less than 7%, less than 6.9%, less than 6.8%, less than 6.7%, less than 6.6%, less than 6.5%, less than 6.4%, less than 6.3%, less than 6.2%, less than 6.1%, less than 6%, less than 5.9%, less than 5.8%, less than 5.7%, less than 5.6%, less than 5.5%, less than 5.4%, less than 5.3%, less than 5.2%, less than 5.1%, less than 5%, less than 4.9%, less than 4.8%, less than 4.7%, less than 4.6%, less than 4.5%, less than 4.4%, less than 4.3%, less than 4.2%, less than 4.1%, less than 4%, less than 3.9%, less than 3.8%, less than 3.7%, less than 3.6%, less than 3.5%, less than 3.4%, less than 3.3%, less than 3.2%, less than 3.1%, less than 3%, less than 2.9%, less than 2.8%, less than 2.7%, less than 2.6%, less than 2.5%, less than 2.4%, less than 2.3%, less than 2.2%, less than 2.1%, less than 2%, less than 1.5%, or less than 1%. In some embodiments, the moisture content is at least 5%, at least 4.9%, at least 4.8%, at least 4.7%, at least 4.6%, at least 4.5%, at least 4.4%, at least 4.3%, at least 4.2%, at least 4.1%, at least 4%, at least 3.9%, at least 3.8%, at least 3.7%, at least 3.6%, at least 3.5%, at least 3.4%, at least 3.3%, at least 3.2%, at least 3.1%, at least 3%, at least 2.9%, at least 2.8%, at least 2.7%, at least 2.6%, at least 2.5%, at least 2.4%, at least 2.3%, at least 2.2%, at least 2.1%, at least 2%, at least 1.5%, at least 1%, at least 0.5%, or at least 0.1%. In some embodiments, microbial compositions described herein comprise a moisture content of between 1-10%, 2-8%, 2-6%, 2.5-5.8%, or 2.8-5.6%.

In some embodiments, methods are provided for generating a composition of a specified moisture content, e.g., by lyophilization. As noted above, compositions described herein may have additional advantages of prolonged shelf life over other microbial compositions.

The viability of the microbes as incorporated in the compositions herein can be at least 5%, at least 10%, at least 20%, at least 30%, at least 40%, at least 50%, at least 60%, at least 70%, at least 80%, at least 90%, at least 95%, at least 97%, at least 99%, at least 99.5%, or at least 99.5%. Further, the viability of the microbial species in the composition can remain stable over the entire shelf-life of the composition until the same can be administered to a subject.

In the case of certain microbial compositions, it can be desired to maintain at least a set number of viable bacteria (e.g., CFU) over a period of time. By way of example, in some cases, a composition herein can maintain at least 10\hat{ }5 CFU, at least 10\hat{ }6 CFU, at least 10\hat{ }7 CFU, at least 10\hat{ }8 CFU, at least 10\hat{ }9 CFU, at least 10\hat{ }10 CFU, at least 10\hat{ }11CFU, at least 10\hat{ }12 CFU, at least 10\hat{ }13 CFU, 10\hat{ }14 CFU, at least 10\hat{ }15 CFU, at least 10\hat{ }16 CFU, at least 10\hat{ }17 CFU, at least 10\hat{ }18 CFU per unit measure, or greater, at the end of the given time window.

A composition as described herein can retain at least a given percentage of the viable microbes in the composition over a given window of time when stored at a particular condition, in some cases at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.9%, or at least 99.95% of the viable microbes over a given time window when stored at a given condition.

A composition as described herein can lose less than a given percentage of the viable microbes in the composition over a given window of time when stored at a particular condition, in some cases less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.2%, less than 0.15%, less than 0.1%, less than 0.09%, less than 0.08%, less than 0.07%, less than 0.06%, less than 0.05%, less than 0.04%, less than 0.03%, less than 0.02%, or less than 0.01% of the viable microbes over a given time window when stored at a given condition.

Some compositions can be formulated such that the viability of the microbes in the composition remains stable over at least 10 days. In some cases, the viability of the microbes in the composition can remain stable over at least 10, 30, 60, 90, 220, 150, 180, 210, 240, 270, 300, 360, or more days. In some cases, the retained viability can be over one or more time windows that may be from 7 days to 24 months, while in some cases, it can be at least one week, at least two weeks, at least 1 month, at least 2 months, at least 3 months, at least 4 months, at least 5 months, at least 6 months, at least 7 months, at least 8 months, at least 9 months, at least 12 months, at least 18 months, at least 24 months, at least 36 months, at least 48 months, or at least 60 months.

A composition, microbial composition, microbe, or strain of interest of the disclosure can be stable at over time when stored under certain conditions. Conditions can comprise temperature conditions, e.g., frozen, cold, or room temperature. Such conditions may include, for example, storage at a temperatures of about −80° C. or less, about −20° C. or less, about −4° C. or less, about 0° C. or less, about 4° C. or less, about 10° C. or less, about 15° C. or less, about 20° C. or less, about 25° C. or less, about 30° C. or less, about 35° C. or less, or about 37° C. or less.

In some cases, a composition of the disclosure can be stable over time when stored at temperatures greater than about −80° C., greater than about −20° C., greater than about −4° C., greater than about 0° C., greater than about 4° C., greater than about 10° C., greater than about 15° C., greater than about 20° C., greater than about 25° C., greater than about 30° C., greater than about 35°, or greater than about 37° C.

In some cases, a composition of the disclosure can be stable over time when stored at a temperature of about −80° C. to 37° C., −80° C. to 35° C., −80° C. to 30° C., −80° C. to 25° C., −80° C. to 20° C., −80° C. to 15° C., −80° C. to 10° C., −80° C. to 4° C., −80° C. to 0° C., −80° C. to −4° C., −80° C. to −20° C., −20° C. to 37° C., −20° C. to 35° C., −20° C. to 30° C., −20° C. to 25° C., −20° C. to 20° C., −20° C. to 15° C., −20° C. to 10° C., −20° C. to 4° C., −20° C. to 0° C., −20° C. to −4° C., −4° C. to 37° C., −4° C. to 35° C., −4° C. to 30° C., −4° C. to 25° C., −4° C. to 20° C., −4° C. to 15° C., −4° C. to 10° C., −4° C. to 4° C., −4° C. to 0° C., 0° C. to 37° C., 0° C. to 35° C., 0° C. to 30° C., 0° C. to 25° C., 0° C. to 20° C., 0° C. to 15° C., 0° C. to 10° C., 0° C. to 4° C., 4° C. to 37° C., 4° C. to 35° C., 4° C. to 30° C., 4° C. to 25° C., 4° C. to 20° C., 4° C. to 15° C., 4° C. to 10° C., 10° C. to 37° C., 10° C. to 35° C., 10° C. to 30° C., 10° C. to 25° C., 10° C. to 20° C., 10° C. to 15° C., 15° C. to 37° C., 15° C. to 35° C., 15° C. to 30° C., 15° C. to 25° C., 15° C. to 20° C., 20° C. to 37° C., 20° C. to 35° C., 20° C. to 30° C., 20° C. to 25° C., 25° C. to 37° C., 25° C. to 35° C., 25° C. to 30° C., 30° C. to 37° C., 30° C. to 35° C., or 35° C. to 37° C.

By way of example, in some cases, the storage temperature for these compositions may be about −80° C., about −20° C., about −4° C., about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C.

In some cases, improved viability, stability, or shelf life can be achieved through the elimination of certain materials from the production process used to generate dried microbial compositions. In some cases, removing or reducing animal products, animal by-products, dairy-derived components, animal-derived components, or a combination thereof from the processing of microbial compositions can improve the viability, stability, or shelf life of the microbial compositions provided herein, e.g., removing or reducing animal products in growth media or skim milk in cryoprotectants. For example, using growth media that is free or substantially free of animal products can improve the viability, stability, or shelf life of a microbial composition provided herein. In some cases, use of a cryoprotectant that is dairy-free or substantially free of dairy-derived components can enhance the shelf life of dry powder formulations of the microbial strains described herein.

### IV. Methods of Treating, Ameliorating, or Mitigating Health Conditions

The disclosure provides a method for administering a composition to a subject. Altering the composition of a microbiome in a subject can have desired health consequences. Compositions of the disclosure can be administered as a therapeutic and/or a cosmetic for treating, ameliorating, or mitigating a health condition. Compositions designed to alter the host microbiome(s) can result in a reduction of patient symptoms, prevention of disease, and/or treatment of the disease or health condition.

The disclosure provides methods for the restoration of a microbial habitat of a subject to a healthy state. The method can comprise microbiome correction and/or adjustment including for example, administering beneficial microbes, replenishing native microbes, removing pathogenic microbes, administering prebiotics, administering growth factors necessary for microbiome survival, or a combination thereof. In some embodiments, the method may also comprises administering antimicrobial agents such as antibiotics.

In some cases, a composition as described herein can be used to treat, ameliorate, or mitigate a gut health condition or gut-oriented disorder as described herein. In such cases, the composition can be animal product-free, animal by-product-free, dairy-free, free of animal-derived components, substantially animal product-free, substantially animal by-product-free, substantially free of dairy-derived components, substantially free of animal-derived components, or a combination thereof.

In some cases, a composition which is dairy-free or substantially free of dairy-derived components can be better tolerated by a subject having a health condition compared to a composition that contains dairy-derived components. In some cases, a composition which is animal product-free or substantially animal product-free can be better tolerated by a subject having a health condition compared to a composition that contains animal products. In some cases, a composition which is animal by-product-free or substantially animal by-product-free can be better tolerated by a subject having a health condition compared to a composition that contains animal by-products. In some cases, a composition which is free or substantially-free of animal-derived components can be better tolerated by a subject having a health condition compared to a composition that contains animal-derived components.

In some cases, a composition which is dairy free or substantially free of dairy-derived components can be better tolerated by a subject having a gut-oriented disorder compared to a composition that contains dairy-derived components. In some cases, a composition which is animal product-free or substantially animal product-free can be better tolerated by a subject having a gut-oriented disorder compared to a composition that contains animal products. In some cases, a composition which is animal by-product-free or substantially animal by-product-free can be better tolerated by a subject having a gut-oriented disorder compared to a composition that contains animal by-products. In some cases, a composition which is free or substantially-free of animal-derived components can be better tolerated by a subject having a gut-oriented disorder compared to a composition that contains animal-derived components.

A composition can be better tolerated than a second composition if it produces fewer side effects than the second composition, has a side effect which is less severe than the same side effect caused by the second composition, produces side effects which are less undesirable than the second composition, or produces no side effects while the second composition may produce side effects.

In some cases, for example, a composition which is dairy-free can be better tolerated in a lactose intolerant subject than is a composition that contains dairy-derived components. In such cases, a subject can experience, for example, less abdominal pain, gas, diarrhea, bloating, or other side effects when a dairy-free composition is administered than when a composition is administered that contains dairy-derived components.

The microbial compositions and formulations comprising those compositions, as described herein, can comprise microbial species described herein. Any of these microbes can be used for treating, ameliorating, or mitigating a variety of disorders, as a formulation comprising such microbes, alone or in combination. Some of the disorders that can be treated, ameliorated, or mitigated with these compositions and formulations can be gut-oriented disorders. These can include disorders like gut dysbiosis, IBS/IBD, inflammatory disorders of the gut, dysbiosis-associated disorders, and other disorders that may have a symptom related to gut-microbial-misbalance. Additional disorders which can be treated, ameliorated, or mitigated using these microbes can include metabolic disorders, including diabetes and prediabetes.

In some embodiments, the disclosure provides a microbial composition that is used to manage blood sugar and type 2 diabetes. The composition can be for the dietary management of type 2 diabetes. The composition can comprise media, probiotics. The composition can manage healthy A1C and blood glucose levels. The composition can result in a statistically significant reduction in HbA1C and blood sugar spikes in people with type 2 diabetes in a randomized, double-blinded, placebo-controlled clinical trial across multiple sites in the United States.

A. Microbes in The Composition

A microbe of the disclosure can comprise an rRNA comprising at least about 85%, 90%, 95%, 98%, 99%, 99.5%, or 100% sequence identity to the 16S rRNA and/or 23S rRNA of Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, or Roseburia intestinalis.

A microbe of the disclosure can comprise an rRNA comprising at least about 85%, 90%, 95%, 98%, 99%, 99.5%, or 100% sequence identity to the 16S rRNA and/or 23S rRNA of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii.

In some embodiments, a composition can comprise one or more microbes comprising an rRNA comprising at least about 85%, 90%, 95%, 98%, 99%, 99.5%, or 100% sequence identity to the 16S rRNA and/or 23S rRNA of Akkermansia muciniphila, Bifidobacterium infantis, Clostridium beijerinckii, Clostridium butyricum, Eubacterium hallii, or any combination thereof.

In some embodiments, a composition can comprise one or more microbes comprising an rRNA comprising at least about 85%, 90%, 95%, 98%, 99%, 99.5%, or 100% sequence identity to the 16S rRNA and/or 23S rRNA of Akkermansia muciniphila, Bifidobacterium infantis, Eubacterium hallii, or any combination thereof.

In some embodiments, a composition can comprise one or more microbes comprising an rRNA comprising at least about 85%, 90%, 95%, 98%, 99%, 99.5%, or 100% sequence identity to the 16S rRNA and/or 23S rRNA of Akkermansia muciniphila and/or Eubacterium hallii.

In some embodiments, a composition can comprise of an rRNA comprising at least about 85%, 90%, 95%, 98%, 99%, 99.5%, or 100% sequence identity to the 16S rRNA and/or 23S rRNA of Akkermansia muciniphila, and Eubacterium hallii. In some embodiments, the composition comprises Akkermansia muciniphila, and Eubacterium hallii.

In some embodiments, a composition can comprise one or more microbes comprising an rRNA comprising at least about 85%, 90%, 95%, 98%, 99%, 99.5%, or 100% sequence identity to the 16S rRNA and/or 23S rRNA of Clostridium butyricum, Clostridium beijerinckii, Bifidobacterium infantis, or any combination thereof. In some embodiments, the composition comprises Clostridium butyricum, Clostridium beijerinckii, and Bifidobacterium infantis.

In some cases, a microbial strain can be live. In some cases, a microbial strain can be viable. In some cases, a microbial strain may not be a spore. In some cases, the microbe can be an anaerobic microbe (e.g., a facultative anaerobe, an obligate anaerobe, or an aerotolerant anaerobe).

In some embodiments, microbial compositions for administration to a subject having lactose intolerance can comprise microbial strains of interest with at least about 70%, 75%, 80%, 85%, 90%, 95%, 98%, 99%, 99.5% or 100% sequence identity to the 16S rRNA and/or 23S rRNA of one or more strains of an isolated and purified microbe selected from the group consisting of Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. In some cases, a microbial strain can be an anaerobic microbe. In some cases, the strain or strains can be live. In some cases, the strain or strains can be viable.

In some embodiments, microbial compositions for administration to a subject having a gut-oriented disorder can comprise microbial strains of interest with at least about 70%, 75%, 80%, 85%, 90%, 95%, 98%, 99%, 99.5% or 100% sequence identity to the 16S rRNA and/or 23 S rRNA of Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, or any combination thereof.

The microbial compositions described herein may include one or more microbial populations of microbial species selected from Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, or Roseburia intestinalis.

In some cases, a microbial composition may not comprise a Lactobacillus strain.

Where more than one microbial population is present, each of the populations may be present in the composition in the same amount or in any range of different amounts. For example, the ratio of any two microbial populations in a composition as described herein can be about 1:1, 1:2, 1:5, 1:10, 1:25, 1:50, 1:100, 1:1000, 1:10,000, or 1:100,000 by weight. As another example, the ratio of two microbial populations in a composition as described herein can be about 1:1, 1:2, 1:5, 1:10, 1:25, 1:50, 1:100, 1:1000, 1:10,000, or 1:100,000 by cell count (e.g., viable CFU). As another example, the ratio of two microbial populations in a composition as described herein can be about 1:1, 1:2, 1:5, 1:10, 1:25, 1:50, 1:100, 1:1000, 1:10,000, or 1:100,000 by volume.

In some embodiments, the microbial compositions may comprise a combination of any one or more microbes described herein. In particular, in many cases, the compositions described herein may include at least one microbial population selected from the microbial species herein in combination with at least one other microbial population of a different species. In certain cases, the compositions may include one, two, three, four, five, six, seven, eight, nine, ten, or more microbial populations selected from the microbial species disclosed herein. Examples of compositions including combination microbial populations for different applications are described in, for example, U.S. Pat. No. 9,486,487, the full disclosure of which is hereby incorporated herein by reference in its entirety for all purposes.

In some embodiments, a microbial composition comprises Clostridium beijerinckii WB-STR-0005, Clostridium butyricum WB-STR-0006, Bifidobacterium infantis 100, Akkermansia muciniphila WB-STR-0001, Eubacterium hallii WB-STR-0008), or a combination thereof. In some embodiments, a microbial composition comprises Clostridium beijerinckii WB-STR-0005, Clostridium butyricum WB-STR-0006, Bifidobacterium infantis 100, Akkermansia muciniphila WB-STR-0001, and Eubacterium hallii WB-STR-0008).

In some embodiments, the composition's precise strains of probiotics and prebiotics restore the body's natural ability to metabolize fiber and regulate blood sugar.

B. Formulations

According to the disclosure, the composition can be formulated in a variety of forms, like, for example, a powder, tablet, enteric-coated capsule (e.g. for delivery to ileum/colon), or pill that can be administered to an individual by any suitable route.

A dry, powdered form of the composition can help maintain stability of the composition (e.g., as it has reduced moisture content), without having any effect on the viability of the microbes in the composition.

In an embodiment of the disclosure, the lyophilized formulation can be mixed with a pre-defined quantity of saline or other additives prior to administration. This composition can be administered orally, for example, in the form of a suspension.

Alternatively, in another embodiment, the composition can be administered orally in the form of a pill with an enteric coating that refrains the pill from disintegrating before it reaches the intestine (e.g., for delivery to ileum or colon) of the individual. In still other aspects, the compositions described herein may be combined with other ingredients to provide alternative formats, e.g., food forms such as bars, lozenges, chewables, and the like.

In some cases, a composition can be provided as a liquid beverage or a semi-solid paste.

The compositions described herein can be provided in any suitable administrable format. For instance, it can be delivered orally, parenterally, by venous routes, or as a suppository. The composition may also be enterically coated with a coating layer that does not let the composition disintegrate anywhere in the body of the individual other than the part of the intestine it can be directed to—which may be the stomach, colon, ileum, duodenum, jejunum, or any other part of the small intestine.

In some embodiments, the microbial composition may comprise additional functional components such as, for example, prebiotic materials. In some embodiments, the prebiotic can be inulin, which can serve as an energy source for the microbes in the composition once administered.

In some embodiments, a composition of the disclosure (e.g., a microbial composition) is provided in capsule form (for example, as a package of 60 capsules). In some embodiments, the composition is perishable and is to be kept refrigerated. In some embodiments, the composition is best if used within 2 months of opening. The composition can be vegan.

Microbial compositions as disclosed herein can be formulated as a supplement, for example, a dietary supplement (e.g., nutritional supplement), or a daily supplement. A dietary supplement can be a product that is taken by mouth that contain a dietary ingredient used to supplement the diet. A dietary supplement can be intended to provide nutrients that may otherwise not be consumed in sufficient quantities; for example, vitamins, minerals, proteins, amino acids or other nutritional substances. In some embodiments, a dietary supplement is not intended to treat, diagnose, cure, or alleviate the effects of a disease or condition. A dietary supplement can be in any form disclosed herein.

The composition can be formulated in a variety of food formats, such as in a bar, a chewable, gummy, gum, candy, or in an effervescent (e.g., fizzable) form such as powder or tablet. Non-limiting examples of foods and drinks that can incorporate the microbial compositions include, for example, bars, shakes, juices, beverages, frozen food products, fermented food products, and soy-based products. All of these may be formulated without any animal-derived components to suit the dietary needs and tolerance needs of gut-oriented disorder patients, for example, free or substantially-free of animal products, animal by-products, dairy-derived components, animal-derived components, or a combination thereof.

Microbial compositions as disclosed herein can be formulated as a medical food. Microbial compositions as disclosed herein can be labeled as a medical food. A medical food can be a food which is formulated to be consumed or administered enterally under the supervision of a physician and which is intended for the specific dietary management of a disease or condition (e.g., a disease or condition disclosed herein), for which distinctive nutritional requirements, based on recognized scientific principles, are established by medical evaluation. In some embodiments, medical foods can be distinguished from the broader category of foods for special dietary use, for example, by the requirement that medical foods are intended to meet distinctive nutritional requirements of a disease or condition, are intended to be used under medical supervision, and are intended for the specific dietary management of a disease or condition. The supervision of a physician can refer to ongoing medical supervision (e.g., in a health care facility or as an outpatient) by a physician who has determined that the medical food is necessary to the subject's overall medical care. The subject can generally see the physician on a recurring basis for, among other things, instructions on the use of the medical food as part of the dietary management of a given disease or condition.

In some embodiments, medical foods are not those simply recommended by a physician as part of an overall diet to manage the symptoms or reduce the risk of a disease or condition. Rather, in some embodiments, medical foods can be foods that are specially formulated and processed (as opposed to a naturally occurring foodstuff used in a natural state) for a subject who requires use of the product, for example, as a major component of a disease or condition's specific dietary management. In some embodiments, medical foods are not regulated as drugs, and do not require a prescription. A medical food can be in any form disclosed herein.

In some embodiments, a composition of the disclosure is a medical food that is used only under medical supervision. In some embodiments, the composition is used to manage blood sugar and type 2 diabetes. In some embodiments, the composition is for the dietary management of type 2 diabetes. In some embodiments, the composition is a composition of medical probiotics. In some embodiments, the composition manages healthy A1C and blood glucose levels. In some embodiments, the composition results in a statistically significant reduction in HbA1C and blood sugar spikes in people with type 2 diabetes in a randomized, double-blinded, placebo-controlled clinical trial across multiple sites in the United States.

An enteric coating can protect the contents of the oral formulation, for example, from the acidity of the stomach, and provide delivery to the ileum and/or upper colon regions. Non-limiting examples of enteric coatings include pH sensitive polymers (e.g., Eudragit FS30D), methyl acrylate-ethacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some embodiments, the enteric coating can be formed by a pH sensitive polymer. In some embodiments, the enteric coating can be formed by eudragit FS30D.

The enteric coating can be designed to dissolve at any suitable pH. In some embodiments, the enteric coating can be designed to dissolve at a pH greater than about pH 6.5 to about pH 7.0 . In some embodiments, the enteric coating can be designed to dissolve at a pH greater than about pH 6.5 . In some embodiments, the enteric coating can be designed to dissolve at a pH greater than about pH 7.0 . In some cases, the enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

Preservatives can be used in microbial compositions. Examples of such preservatives include, but are not limited to, sorbic acid, sorbates, parabens, lactic acid, benzoates, propionic acid, sodium sorbate. A combination of more than one preservative May also be used. In embodiments, compositions or formulations described herein may contain a preservative. In further embodiments, the amount of preservative present in any composition or formulation is sufficient to act a preservative of the composition or formulation, thereby enhancing the stability and/or survival one or more components of the composition or formulation.

Formulations provided herein can include the addition of one or more agents to the therapeutics or cosmetics in order to enhance stability and/or survival of the microbial formulation. Non-limiting examples of stabilizing agents include genetic elements, glycerin, ascorbic acid, lactose, tween, alginate, xanthan gum, carrageenan gum, mannitol, palm oil, and poly-L-lysine (POPL).

In the present disclosure, the strains of interest may be genetically modified in some embodiments. In other embodiments, one or more strains of interest are not modified or recombinant (e.g., non-GMO). In some embodiments, all of the strains are not genetically modified (e.g., non-GMO). In some embodiments, the formulation comprises microbes that can be regulated, for example, a microbe comprising an operon or promoter to control microbial growth. Microbes of the disclosure can be produced, grown, or modified using any suitable methods, including recombinant methods.

A therapeutic or cosmetic composition can include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, antioxidants, gums, coating agents, coloring agents, flavoring agents, dispersion enhancer, disintegrant, coating agents, plasticizers, preservatives, suspending agents, emulsifying agents, plant cellulosic material and spheronization agents, and any combination thereof.

A therapeutic or cosmetic composition can be substantially free of preservatives. In some applications, the composition may contain at least one preservative.

In some embodiments, a composition of the disclosure comprises as ingredients, Probiotic Blend (Clostridium beijerinckii WB-STR-0005, Clostridium butyricum WB-STR-0006, Bifidobacterium infantis 100, Akkermansia muciniphila WB-STR-0001, Eubacterium hallii WB-STR-0008), Chicory Inulin and Oligofructose, Fruit and Vegetable Juice (Color), Magnesium Stearate, Capsule (Water, Hydroxylpropyl Methylcellulose Phthalate, Hydroxypropyl Methylcellulose).

Compositions of the disclosure can be administered in combination with another therapy, for example, immunotherapy, chemotherapy, radiotherapy, anti-inflammatory agents, anti-viral agents, anti-microbial agents, and anti-fungal agents.

Compositions of the disclosure can be packaged as a kit. In some embodiments, a kit includes written instructions on the administration/use of the composition. The written material can be, for example, a label. The written material can suggest conditions and/or methods of administration. The instructions can provide the subject and the supervising physician with guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

Pharmaceutical compositions of the disclosure can be formulated with any suitable therapeutically-effective concentration of prebiotic. In some embodiments, the prebiotic can be inulin. For example, the therapeutically-effective concentration of a prebiotic can be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a pharmaceutical composition can be about 70 mg/ml.

As a further example, the therapeutically-effective concentration of a prebiotic can be at least about 1 mg/g, about 2 mg/g, about 3 mg/g, about 4 mg/g, about 5 mg/g, about 10 mg/g, about 15 mg/g, about 20 mg/g, about 25 mg/g, about 30 mg/g about 35 mg/g, about 40 mg/g, about 45 mg/g, about 50 mg/g, about 55 mg/g, about 60 mg/g about 65 mg/g, about 70 mg/g, about 75 mg/g, about 80 mg/g, about 85 mg/g, about 90 mg/g, about 95 mg/g, about 100 mg/g, about 110 mg/g, about 125 mg/g, about 130 mg/g, about 140 mg/g, or about 150 mg/g. For example, the therapeutically-effective concentration of a prebiotic can be at most about 1 mg/g, about 2 mg/g, about 3 mg/g, about 4 mg/g, about 5 mg/g, about 10 mg/g, about 15 mg/g, about 20 mg/g, about 25 mg/g, about 30 mg/g, about 35 mg/g, about 40 mg/g, about 45 mg/g, about 50 mg/g, about 55 mg/g, about 60 mg/g, about 65 mg/g, about 70 mg/g, about 75 mg/g, about 80 mg/g, about 85 mg/g, about 90 mg/g, about 95 mg/g, about 100 mg/g, about 110 mg/g, about 125 mg/g, about 130 mg/g, about 140 mg/g, or about 150 mg/g. For example, the therapeutically-effective concentration of a prebiotic can be about 1 mg/g, about 2 mg/g, about 3 mg/g, about 4 mg/g, about 5 mg/g, about 10 mg/g, about 15 mg/g, about 20 mg/g, about 25 mg/g, about 30 mg/g, about 35 mg/g, about 40 mg/g, about 45 mg/g, about 50 mg/g, about 55 mg/g, about 60 mg/g, about 65 mg/g, about 70 mg/g, about 75 mg/g, about 80 mg/g, about 85 mg/g, about 90 mg/g, about 95 mg/g, about 100 mg/g, about 110 mg/g, about 125 mg/g, about 130 mg/g, about 140 mg/g, or about 150 mg/g.

C. Dosing

The microbial compositions described herein may be administrated once or multiple times daily, every other day, one/two/three times a week, or at other appropriate intervals for treating, ameliorating, or mitigating a disorder as described herein. Pharmaceutical compositions of the disclosure can be administered, for example, 1, 2, 3, 4, 5, or more times daily, every other day, three times a week, every week, every two weeks, every three weeks, every four weeks, every five weeks, every six weeks, every eight weeks, every 10 weeks, monthly, every two months, every three months, every four months, every five months, every six months, every eight months, every 10 months, every 12 months, or at such other intervals as are warranted.

In some embodiments, the microbial formulation can be administered before, during, and/or after food intake by a subject. In some embodiments, the formulation is administered with food intake by the subject. In some embodiments, the formulation can be administered simultaneously with food intake.

The appropriate quantity of a therapeutic or cosmetic composition to be administered, the number of times a composition is administered, and unit dose can vary according to a subject and/or the disease state of the subject.

Pharmaceutical compositions described herein can be in unit dosage forms suitable for single administration of precise dosages. In unit dosage form, the formulation can be divided into unit doses containing appropriate quantities of one or more microbial compositions. The unit dosage can be in the form of a package containing discrete quantities of the formulation. Non-limiting examples are pills, tablets, capsules, and liquids in vials or ampoules. Aqueous suspension compositions can be packaged in single-dose non-recloseable containers. The composition can be in a multi-dose format. Multiple-dose recloseable containers can be used, for example, in combination with a preservative. Formulations for parenteral injection can be presented in unit dosage form, for example, in ampoules, or in multi-dose containers with a preservative.

The dosage can be in the form of a solid, semi-solid, or liquid composition. Non-limiting examples of dosage forms suitable for use in the disclosure include feed, food, pellet, lozenge, liquid, elixir, aerosol, inhalant, spray, powder, fizz-able powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

In some embodiments, a composition of the disclosure is taken daily with food, as 1 pill in the morning and 1 pill in the evening.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### EXAMPLES

### Example 1: Cells Remain Viable When Freeze Dried Using a Dairy Free Cryoprotectant

This example describes an illustrative microbial composition exhibiting enhanced maintenance of viability over time compared with a control microbial composition. Illustrative microbes can include Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and Faecalibacterium prausnitzii.

A population of cultured microbes was freeze dried. During the lyophilization step of the freeze-drying protocol, a dairy-free cryoprotectant containing no dairy components or dairy-derived components was used to maintain viability of the cells. As a control, an additional population of cells was freeze dried using the same protocol as the experimental group. In the control group, during the lyophilization step of the freeze-drying protocol, a cryoprotectant comprising dairy-derived components (skim milk) was used.

Freeze dried cells were stored as a dry powder at room temperature. After the passage of a predetermined number of days, cells from the experimental and control compositions were rehydrated, serial dilutions were plated on agar, and plates were incubated overnight at 37° C. The next morning, colony forming units were counted as a measure of cell viability over time.

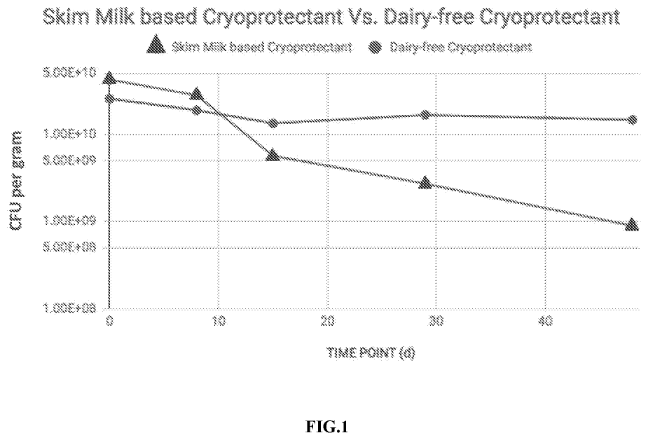

FIG. 1 shows the viability of cells freeze-dried in a dairy-free cryoprotectant vs a cryoprotectant comprising skim milk over a period of 50 days. Data are presented as the amount of viable cells in CFU/g present over time in days. Over the course of the experiment, the composition which had been lyophilized in the skim milk cryoprotectant exhibited an approximately 50-fold reduction in viability. In contrast, the composition which had been lyophilized in the dairy free cryoprotectant maintained viability over time and did not exhibit any significant reduction in cell viability over the course of the experiment.

### Example 2: Increasing Viability of Microbial Composition Substantially Free of Animal Derived Components

This example describes an illustrative microbial composition lacking animal-derived components that exhibits enhanced maintenance of viability over time compared with a control microbial composition.

A microbial composition is produced by culturing, harvesting, and freeze-drying a microbial strain of interest (e.g., Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, or Faecalibacterium prausnitzii). The steps involved in producing the microbial composition are performed without use of any animal-derived components. The culturing is performed using vegetable-based or yeast-based media. During the lyophilization step of the freeze-drying protocol, an animal product-free and dairy-free cryoprotectant is used to maintain viability of the cells.

As a control, an additional population of cells is produced that is cultured in animal media and freeze dried using a cryoprotectant that comprises animal products and/or dairy-derived components.

Freeze-dried cells are stored as a dry powder at room temperature. After the passage of a predetermined number of days, cells from the experimental and control compositions are rehydrated, serial dilutions are plated on agar, and plates are incubated overnight at 37° C. The next morning, colony forming units are counted as a measure of cell viability over time. Viability of the cells produced in the absence of animal derived products is measured, and compared to viability of control cells. Data is presented as the amount of viable cells in CFU/g over a period of days. Cells that lack animal-derived products exhibit enhanced maintenance of viability over time compared to the control cells.

### Example 3: Increased Viability of Microbial Population When Cryoprotected Using Dairy Free Combination of Lactate and Trehalose

This example provides an illustrative increase in viability of a microbial population when cryoprotected using a dairy free combination of lactate and trehalose compared to cryoprotection using skim milk.

A population of E. hallii was cultured on a vegetable media and was freeze dried. During the lyophilization step of the freeze-drying protocol, a dairy-free cryoprotectant containing no dairy components or dairy-derived components was used to maintain viability of the cells. The dairy-free cryoprotectant comprises a combination of sodium lactate and trehalose. The dairy-free cryoprotectant was added to the bacteria at 20% weight by volume. As a control, E. hallii cultured on the vegetable media was freeze-dried using 5% weight by volume skim milk as a cryoprotectant.

Both types of freeze-dried cells were then stored as a dry powder at room temperature. After the passage of a predetermined number of days, cells from the experimental and control compositions were rehydrated, serial dilutions were plated on agar, and plates were incubated overnight at 37° C. The next morning, colony forming units were counted as a measure of cell viability over time.

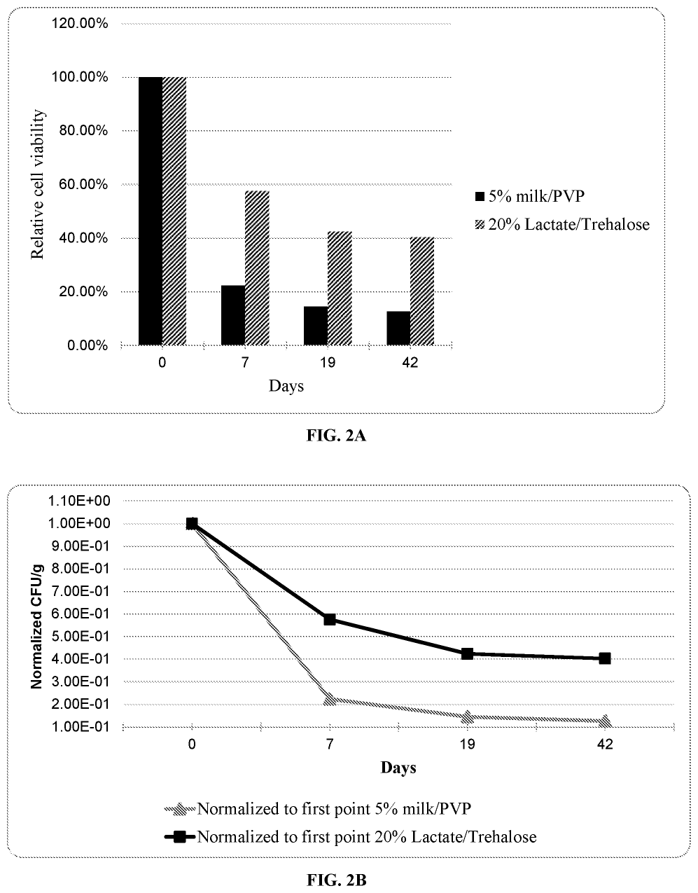

FIG. 2A shows the percent viability of E. hallii freeze-dried in the presence of lactate-trehalose cryoprotectant vs a cryoprotectant comprising skim milk over a period of 42 days. FIG. 2B provides the normalized data used to generate FIG. 2A. Data are presented as the amount of viable cells in CFU/g along the Y-axis present over time in days along the X-axis. Over the course of the experiment, the composition which had been lyophilized in the skim milk cryoprotectant retained only 10% viability. In contrast, the composition which had been lyophilized in lactate-trehalose cryoprotectant maintained at least 40% viability over the course of the experiment.

### Example 4: Increased Stability of Microbial Populations When Cryoprotected Using 20% Lactate and Trehalose Compared to Other Cryoprotectants

This example provides an illustrative increase in viability of a microbial composition when cryoprotected using a dairy free combination of lactate and trehalose in the presence of a desiccant compared to when the microbial composition was cryoprotected using milk/polyvinylpyrrolidone (PVP), or lactate, with or without a desiccant.

0.1 g of a population of E. hallii was cultured on a vegetable media and was freeze dried. During the lyophilization step of the freeze-drying protocol, various cryoprotectants were used to enhance viability of the cells. In one sample, a combination of sodium lactate and trehalose was added to the bacteria at 20% weight by volume. A desiccant was also present in the combination. For comparison, additional samples were prepared using 5% milk in polyvinylpyrrolidone (PVP) as a cryoprotectant or 20% lactate as a cryoprotectant. Each of the resulting freeze dried powers was then stored, in the presence or absence of a desiccant at room temperature.

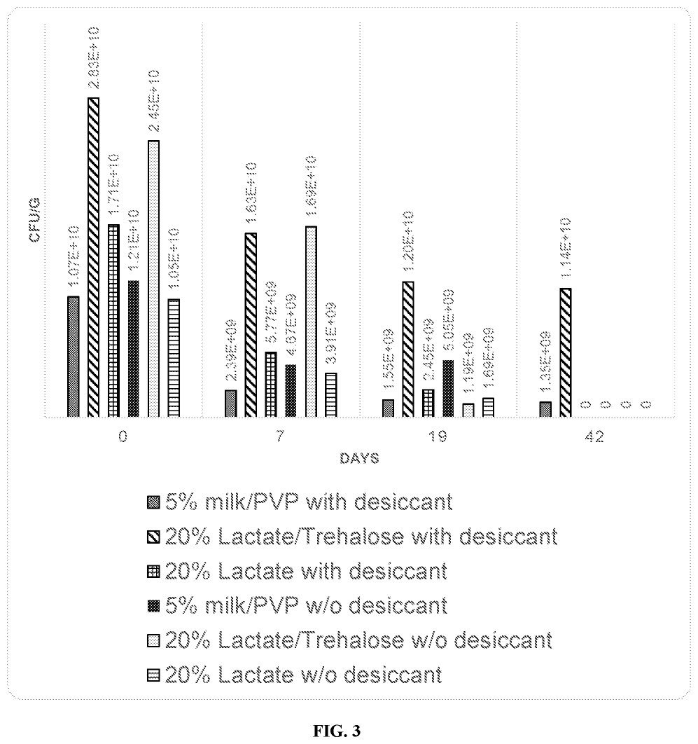

After the passage of a predetermined number of days, cells from each of the six conditions were rehydrated, serial dilutions were plated on agar, and plates were incubated overnight at 37° C. The next morning, colony forming units were counted as a measure of cell viability over time. The results are as depicted in FIG. 3.

FIG. 3 provides the data presented as viable cells in CFU/g along the Y-axis, as measured on the timepoint in days as indicated on the X-axis. Over the course of the experiment, the composition which had been lyophilized in 20% lactate/trehalose and stored in the presence of a desiccant provided the most viable composition, retaining approx. 1.14×10\hat{ }10 CFU/g at 42 days in room temperature. In contrast, the compositions which had been lyophilized and stored as per any of the other 5 conditions had hardly any sample left to test viability, or dropped off close to 0 CFU/g, after 42 days in room temperature.

What is claimed is:

1. A composition comprising at least one powdered microbial population, lactate, and trehalose.

2. The composition of claim 1, wherein the lactate is a lactate salt.

3. The composition of claim 1, wherein the lactate is sodium lactate.

4. The composition of any one of claims 1-3, wherein the lactate and trehalose are present in sufficient amount to act as a cryoprotectant.

5. The composition of any one of claims 1-4, wherein the microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii.

6. The composition of any one of claims 1-5, wherein the lactate and trehalose are present in an amount from 1% to 50% weight by volume.

7. The composition of any one of claims 1-6, wherein the lactate and trehalose are present in at least 5% weight by volume.

8. The composition of any one of claims 1-7, wherein the lactate and trehalose are present at about 20% weight by volume.

9. The composition of any one of claims 1-8, wherein the microbial population is lyophilized.

10. The composition of any one of claims 1-9, wherein the microbial population is viable.

11. The composition of any one of claims 1-10, wherein the microbial population has a viability of at least 1×10\hat{ }5 CFU/g of the composition.

12. The composition of any one of claims 1-11, wherein the composition is dairy-free.

13. The composition of any one of claims 1-12, wherein the composition comprises substantially no animal products.

14. The composition of any one of claims 1-13, wherein the composition comprises an effective amount of a preservative.

15. The composition of any one of claims 1-14, further comprising a desiccant.

16. The composition of claim 15, where the desiccant is selected from the group consisting of, silica gel, clay, and calcium sulfate.

17. The composition of claim 15, wherein the composition has a moisture content from about 2.8% to about 5.6%.

18. The composition of any one of claims 1-14, wherein the composition is a pill, capsule, or tablet.

19. The composition of claim 18, wherein the pill, capsule or tablet is enterically-coated, or wherein the pill, capsule, or tablet disintegrates to release its contents in the small intestine.

20. The composition of any one of claims 1-19, wherein the microbial population maintains at least 50% viability at room temperature for at least 5 days or at least 7 days.

21. The composition of any one of claims 1-20, wherein the microbial population maintains at least 40% viability at room temperature for at least 19 days or at least 42 days.

22. Method of producing a microbial product, the method comprising:
combining a microbial population with lactate and trehalose so as to create a microbial product.

23. The method according to claim 22, further comprising lyophilizing, spray drying, and/or freeze-drying the microbial population.

24. The method according to any one of claims 22-23, wherein the lactate is a lactate salt.

25. The method according to any one of claims 22-24, wherein the lactate is sodium lactate.

26. The method according to any one of claims 22-25, wherein the lactate and trehalose are present in sufficient amount to act as a cryoprotectant.

27. The method according to any one of claims 22-26, wherein the microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii.

28. The method according to any one of claims 22-27, wherein the lactate and trehalose are present in an amount from 1% to 50% weight by volume.

29. The method according to any one of claims 22-28, wherein the lactate and trehalose are present in at least 5% weight by volume.

30. The method according to any one of claims 22-29, wherein the lactate and trehalose are present at about 20% weight by volume.

31. The method according to any one of claims 22-30, wherein the microbial product is dairy-free.

32. The method according to any one of claims 22-31, wherein the microbial product comprises substantially no animal products.
